(19) United States
(12) Patent Application Publication
(10) Pub. No.: US 2025/0354110 A1
(43) Pub. Date: Nov. 20, 2025

(54) METHOD OF PLASTICIZING SILK MATERIALS AND PRODUCT THEREOF

(71) Applicant: Trustees of Tufts College, Medford, MA (US)

(72) Inventors: David L. Kaplan, Concord, MA (US); Chunmei Li, Stoneham, MA (US); Yushu Wang, Medford, MA (US)

(21) Appl. No.: 19/223,953

(22) Filed: May 30, 2025

Related U.S. Application Data
(63) Continuation of application No. PCT/US2023/082533, filed on Dec. 5, 2023.
(60) Provisional application No. 63/386,151, filed on Dec. 5, 2022.

(51) Int. Cl. C12N 5/00; C08J 3/205; C08L 89/00

(52) U.S. Cl. CPC C12N 5/0068; C08J 3/2053; C08L 89/00; C08J 2389/00; C08L 2205/06; C12N 2533/50

(57) ABSTRACT

Disclosed herein are methods of making a silk fibroin article including mist-plasticizing a lyophilized silk fibroin powder, thereby producing a modified powder, wherein the mist-plasticizing comprises exposing the lyophilized silk fibroin powder to a mist of an aqueous plasticizer composition and thermally compressing the modified powder into a solid form, thereby forming a silk fibroin article.

### CLAIM TO PRIORITY

This application claims benefit of and is a continuation of International Patent Application No. PCT/US2023/082533 (Attorney Docket. No. 2095.0581), filed Dec. 5, 2023, and entitled “METHOD OF PLASTICIZING SILK MATERIALS AND PRODUCT THEREOF,” International Pub. No. WO2024123781, which is hereby incorporated by reference in its entirety for all purposes.

International Patent Application No. PCT/US2023/082533 claims the benefit of the following provisional application, which is hereby incorporated by reference in its entirety for all purposes: U.S. Patent Application Ser. No. 63/386,151 (Attorney Docket No. 2095.0420), filed Dec. 5, 2022.

### STATEMENT REGARDING FEDERALLY FUNDED RESEARCH

This invention was made with government support under grant FA9550-20-1-0363 awarded by the United States Air Force. The government has certain rights in the invention.

### BACKGROUND

Traditional approaches for plasticizing silk material to tune its physical properties are solution-based processes, where the plasticizer is mixed with an aqueous silk solution followed by processing into silk products via techniques such as freeze drying and film casting. Such techniques may not allow for fabrication of new materials formats with versatile control of molecular structure and physical properties of the final silk products.

Silk fibroin can be processed to various material forms for specific applications such as film, fiber, hydrogel, sponge, etc. in its aqueous phase, however, the perishable silk solution needs to be preserved for short term storage. To extend the shelf-life of silk production, silk fibroin can be prepared as amorphous powders and molded at high temperatures of up to 145° C. to prepare a stiffer material with a much more stable, dense, and highly crystalline structure that exhibits excellent machining ability. However, the elevated-temperature procedure presents a risk of denaturing the microbial activity in the living material system, and the increased mechanical rigidity may not be compatible with certain human tissues, constraining its applicability in vivo.

Thus, a new method to obtain silk materials with tunable mechanical properties and stable structure with a moderate processing method to meet more applications is desired.

### SUMMARY

In some aspects, the techniques described herein relate to a method of making a silk fibroin article, the method including: a) mist-plasticizing a lyophilized silk fibroin powder, thereby producing a modified powder, wherein the mist-plasticizing includes exposing the lyophilized silk fibroin powder to a mist of an aqueous plasticizer composition; and b) thermally compressing the modified powder into a solid form, thereby forming the silk fibroin article.

In some aspects, the techniques described herein relate to a silk fibroin material having a solid-state NMR 13C spectrum having a C═O-associated signal with at least some peak splitting and an alanine β-carbon-associated signal with at least some peak splitting, wherein an alpha/RC portion of the alanine β-carbon-associated signal associated with alpha-helix and random coil structures has a peak intensity that is higher than a beta portion of the alanine β-carbon-associated signal associated with beta sheet structures.

In some aspects, the techniques described herein relate to a thermally compressed silk fibroin article formed from a mist-plasticized lyophilized silk fibroin powder.

These and other systems, methods, objects, features, and advantages of the present disclosure will be apparent to those skilled in the art from the following detailed description of the preferred embodiment and the drawings.

Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE FIGURES

The disclosure and the following detailed description of certain embodiments thereof may be understood by reference to the following figures:

FIG. 1a, Schematic illustration showing the fabrication steps of the silk bioplastic by plasticizer-assisted thermal molding methods. SSNMR spectra (FIG. 1b) and structural deconvolution analysis (FIG. 1c) of silk powder, water-plasticized silk powder with 20% of water content (20% WPS), 20% WPS thermal-molded at 60° C. (WS/60° C.), and degummed silk fiber (FIG. 2), respectively.

FIG. 2. SSNMR structural deconvolution analysis of the degummed silk fiber.

FIG. 3: SSNMR spectra of the silk bioplastics prepared at different conditions.

FIG. 4A, FIG. 4B, FIG. 4C, and FIG. 4D present SSNMR structural deconvolution analyses of the silk bioplastics prepared at different conditions.

FIG. 5A presents DSC of the silk powder, FIG. 5b presents TGA of the silk powder, and FIG. 5c presents XRD of the silk powder of the silk powder, all at 20% WPS, WS/60° C.

FIG. 6. XRD curves of the 30% WPS, WS/40° C. and degummed silk fiber.

FIG. 7 (a) Cross-sectional SEM images of the silk powder, 20% WPS, WS/60° C. (FIG. 7b) Trajectory plots of residues 152-586 of silk sequence UniProt ID: P05790 during 200 ns simulation and representative snapshots of simulated protein structures for (i) silk powder, (ii) 20% WPS, and (iii) WS/60° C. Quantitative analysis of protein structures including (FIG. 7c) β-sheet, (FIG. 7d) helix, and (FIG. 7e) random coils and turns for simulated models of silk powder, 20% WPS, and WS/60° C., respectively. In the SEM images, scale bars are 10 μm for silk powder and 20% WPS, 500 nm for WS/60° C., and 200 μm for WS/60° C. insertion, respectively.

FIG. 8. SSNMR structural of the silk/glycerol powder with 10% (10% GS), 20% (20% GS), and 30% (30% GS) of glycerol addition, and 10% GS followed by water plasticization with 20% of water content (GWS), and their corresponded bioplastics prepared by thermal molding at 60° C., termed as 10% GS/60° C., 20% GS/60° C., 30% GS/60° C., and GWS/60° C., respectively.

FIG. 9a. Photographs of flexible silk bioplastics of WS/60° C. with folding, twisting, and bending designs. Scale bar, 10 mm. FIG. 9b, SEM images and insert photographs of the WS/60° C. micropillars, showing microscale machining of the silk plastic. FIG. 9c, Photographs of stiffer WS/60° C. machining with various complex patterns. Scale bar is 10 mm. Tensile stress-strain curves (FIG. 9d), Young's modulus (FIG. 9e), and toughness (FIG. 9f) of hydrated and dry WS/60° C., and TS/60° C., respectively.

FIG. 10. Fluorescence densities of the WS/60° C. micropillar without and with C2C12 cells cultured for two weeks (n=4). Data points are shown as mean±s.d.

### DETAILED DESCRIPTION

Before the present disclosure is described in further detail, it is to be understood that the disclosure is not limited to the particular embodiments described. It is also understood that the terminology used herein is for the purpose of describing particular embodiments only, and is not intended to be limiting. The scope of the present disclosure will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, “silk fibroin” refers to silk fibroin protein whether produced by silkworm, spider, or other insect, or otherwise generated (Lucas et al., Adv. Protein Chem., 13: 107-242 (1958)). Any type of silk fibroin can be used in different embodiments described herein. Silk fibroin produced by silkworms, such as Bombyx mori, is the most common and represents an earth-friendly, renewable resource. For instance, silk fibroin used in a silk film may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants, and variants thereof, that can be used. See, e.g., WO 97/08315 and U.S. Pat. No. 5,245,012, each of which is incorporated herein by reference in their entireties.

Disclosed herein is a new method to prepare a dense and flexible silk bioplastic material with high crystallinity by a plasticizer-assisted thermal molding process. In some embodiments, the method includes hydrating lyophilized silk powder and subjecting it to compression. In an example, the process may include: 1) introducing 10-30% contents of plasticizers to the silk system via the silk solutions used to form the material or homogeneously introducing to lyophilized silk powders (LSPs) after they are formed; and 2) plasticized silk powders are subject to thermal molding to generate transparent and flexible silk materials with high crystallinity.

Four major processing parameters of a plasticizer-assisted thermal molding method can be adjusted to modulate the material properties of the final silk products. The molecular structure of LSP, the plasticizer type or content, the compression temperature, and/or the compression pressure each and in combination were found to control the properties of final silk products in plasticizer-assisted thermal molding. This method allows for the fabrication of new materials formats with versatile control of molecular structure and physical properties of the final silk products. For example, using mist plasticization, direct molding of silk products with complex features and sufficient β-sheet content to attain water stability, such as silk micropillars, can be achieved at temperatures as low as room temperature. Further disclosed herein are thermally compressed silk fibroin articles formed from mist-plasticized lyophilized silk fibroin powder (e.g., silk filaments, silk plates, silk micropillars, bone screws, etc.).

Without wishing to be bound by any particular theory, it was not apparent to the inventors that mist treatment of LSPs would enhance material properties. Similarly, it was not apparent that mist treatment of LSPs could significantly expand the variety of material properties that are achievable via thermal compression of LSPs. When thermal compression of LSPs was first exhibited and the inventors identified some areas where the resulting articles could be improved (e.g., reducing water uptake, improving certain material properties, etc.), mist treatment of LSPs was not among the first options that they pursued as experts, which serves as evidence that mist treatment would not have likely occurred to an individual with non-expert skill.

In an embodiment, a method of making a silk fibroin article may include mist-plasticizing a lyophilized silk fibroin powder (LSPs) thereby producing a modified powder. The lyophilized silk fibroin powder may be produced by lyophilizing a silk fibroin solution. An example process includes freeze-drying and milling silk fibroin solution to obtain lyophilized silk powders (LSPs) containing random coils, α-helix content, B-sheet content, and bound water molecules. The molecular structure of the LSPs can be adjusted to modulate the material properties of the final silk products.

Mist-plasticizing may include exposing the lyophilized silk fibroin powder to a mist of an aqueous plasticizer composition. For example, the aqueous plasticizer composition may be a plasticizer solution including plasticizer in an amount by weight of between 0.1% and 50%. In some embodiments, the plasticizer may be an internal plasticizer, such as glucose or polylysine, and may be grafted to silk molecules by chemical modification. In some embodiments, glycine may be added to silk solution and the resulting lyophilized silk/glycerol material may be used as feeding materials for compression molding. In yet other embodiments, proline and urea may be blended with LSPs and used for compression molding.

Mist-plasticizing may be performed at a temperature of between 0° C. and 25° C. The mist density of the mist-plasticizing step may be selected for a desired material property in the silk fibroin article.

In embodiments, the mist of plasticizer may include free water molecules, glycerol, CaCl₂, or internal plasticizers, such as amino acids. The type or content of plasticizer used in this treatment can be adjusted to modulate the material properties of the final silk products.

Mist-treated LSPs may then be subjected to compression molding to generate plasticized silk materials. The temperature and/or pressure of the compression can be adjusted to modulate the material properties of the final silk products.

The modified powder may be thermally compressed into a solid form, thereby forming a silk fibroin article. In embodiments, thermally compressing may be performed at a temperature of between 1° C. and 165° C., including but not limited to, between 1° C. and 95° C., between 1° C. and 65° C., between 1° C. and 50° C. or between 1° C. and 30° C. In embodiments, thermally compressing may be performed at a pressure of between 100 MPa and 1000 MPa, including but not limited to, 500 MPa to 800 MPa or 600 MPa to 700 MPa. In some embodiments, thermally compressing may be applied for a length of time of between 1 second and 10 minutes, including but not limited to, between 5 seconds and 5 minutes or between 10 seconds and 60 seconds.

Silk fibroin articles produced herein may be reduced in size, such as by using a manual or automated tool (e.g., a lathe, a saw, a drill, a file, sandpaper, or the like).

Silk fibroin materials disclosed herein may have a solid-state NMR ¹³C spectrum having a C═O-associated signal with at least some peak splitting and an alanine β-carbon-associated signal with at least some peak splitting, wherein an alpha/RC portion of the alanine β-carbon-associated signal associated with alpha-helix and random coil structures has a peak intensity that is higher than a beta portion of the alanine β-carbon-associated signal associated with beta sheet structures. In some embodiments, a beta portion of the C═O-associated signal associated with beta sheet structures has a peak intensity that is higher than an alpha/RC portion of the C═O associated signal associated with alpha-helix and random coil structures.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” are used as equivalents and may be understood to permit standard variation as would be understood by those of ordinary skill in the art; and (v) where ranges are provided, endpoints are included.

Approximately: as used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biocompatible: the term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

Biodegradable: as used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid) (PLA), poly(glycolic acid) (PGA), poly(lactic-co-glycolic acid) (PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

Compaction: as used herein, the term “compaction” refers to a process by which a material progressively loses its porosity due to the effects of loading.

Composition: as used herein, may be used to refer to a discrete physical entity that comprises one or more specified components. In general, unless otherwise specified, a composition may be of any form—e.g., gas, gel, liquid, solid, etc. In some embodiments, “composition” may refer to a combination of two or more entities for use in a single embodiment or as part of the same article. It is not required in all embodiments that the combination of entities result in physical admixture, that is, combination as separate co-entities of each of the components of the composition is possible; however many practitioners in the field may find it advantageous to prepare a composition that is an admixture of two or more of the ingredients in a pharmaceutically acceptable carrier, diluent, or excipient, making it possible to administer the component ingredients of the combination at the same time.

Fusion: as used herein, the term “fusion” refers to a process of combining two or more distinct entities into a new whole.

Hydrophilic: as used herein, the term “hydrophilic” and/or “polar” refers to a tendency to mix with, or dissolve easily in, water.

Hydrophobic: as used herein, the term “hydrophobic” and/or “non-polar”, refers to a tendency to repel, not combine with, or an inability to dissolve easily in, water.

Improve, increase, or reduce: as used herein or grammatical equivalents thereof, indicate values that are relative to a baseline measurement, such as a measurement in a similar composition made according to previously known methods.

Macroparticle: as used herein, the term “macroparticle” refers to a particle having a diameter of at least 1 millimeter. In some embodiments, macroparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer. In some embodiments, a population of particles is considered a population of macroparticles if the mean diameter of the population is equal to or greater than 1 millimeter.

Microparticle: as used herein, the term “microparticle” refers to a particle having a diameter between 1 micrometer and 1 millimeter. In some embodiments, microparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer. In some embodiments, a population of particles is considered a population of microparticles if the mean diameter of the population is between 1micrometer and 1 millimeter.

Nanoparticle: as used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, nanoparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer. In some embodiments, a population of particles is considered a population of nanoparticles if the mean diameter of the population is equal to or less than 1000 nm.

Physiological conditions: as used herein, has its art-understood meaning referencing conditions under which cells or organisms live and/or reproduce. In some embodiments, the term refers to conditions of the external or internal mileu that may occur in nature for an organism or cell system. In some embodiments, physiological conditions are those conditions present within the body of a human or non-human animal, especially those conditions present at and/or within a surgical site. Physiological conditions typically include, e.g., a temperature range of 20-40° C., atmospheric pressure of 1, pH of 6-8, glucose concentration of 1-20 mM, oxygen concentration at atmospheric levels, and gravity as it is encountered on earth. In some embodiments, conditions in a laboratory are manipulated and/or maintained at physiologic conditions. In some embodiments, physiological conditions are encountered in an organism.

Pure: as used herein, a material, additive, and/or entity is “pure” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure.

Reference: as used herein describes a standard or control relative to which a comparison is performed. For example, in some embodiments, a material, article, additive, entity or other sample, sequence or value of interest is compared with a reference or control material, article, additive, entity or other sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Solid form: as is known in the art, many chemical entities (in particular many organic molecules and/or many small molecules) can adopt a variety of different solid forms such as, for example, amorphous forms and/or crystalline forms (e.g., polymorphs, hydrates, solvates, etc). In some embodiments, such entities may be utilized as a single such form (e.g., as a pure preparation of a single polymorph). In some embodiments, such entities may be utilized as a mixture of such forms.

Substantially: as used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

In some embodiments, methods disclosed herein involve the fabrication of amorphous silk nanomaterials (ASN) generated from aqueous silk fibroin solution. ASN may then be treated by hot pressing, leading to fusion and densification of the silk (e.g., into a silk article). The resulting silk bulk material exhibits specific strength higher than that of most natural structural materials and has been shown effective for fabricating silk-based composites. In addition, it is shown that the engineered silk material has thermoforming properties, which allows the materials to be further transformed to desirable shapes under proper conditions. In some embodiments, compositions and methods described herein demonstrate a thermal and pressure-based, time-efficient and controllable method to transform silk fibroin from a silk fibroin material including substantial amounts of amorphous silk fibroin (for example, in powder form) directly to bulk structural material. In some embodiments, methods and compositions described herein may allow for the application of more traditional process and molding techniques to silk materials, where this was not previously successfully employed for silk. Additionally, in some embodiments, processing methods described herein avoid the need for solvent or aqueous approaches, and providing direct routes to transform silk fibroin material into parts. In accordance with various embodiments, methods described herein provide for the transformation of silk fibroin from amorphous materials to a semi-crystalline high-performance structural material through controlled application of heat and pressure. In some embodiments, provided processes induce a conformation transition of silk molecules from random coil to β-sheet. In some embodiments, provided methods include the processing of natural silk fiber into amorphous silk material (e.g., powder) via degumming, silk fibroin solubilization and freeze drying to prepare the proper premolding materials; feeding the amorphous silk material into a predesigned mold; and inducing the conformation and structure change of silk by applying heat and pressure. Additionally, this method can be processed with silk alone, or with the addition of inorganic fillers or second polymers to generate composite devices. In some cases, the methods described herein can include selecting an elevated temperature and an elevated pressure to produce a desired silk fibroin article of a desired crystallinity and desired material properties and then applying that elevated temperature and elevated pressure to a silk fibroin material having substantially amorphous structure. That is, the methods described herein can predictably select and apply temperatures and pressures to produce articles having desired crystallinity and material properties.

### Silk Materials

Any of a variety of silk materials may be used in accordance with various embodiments. In some embodiments, a silk material may be or comprise silk fibroin (e.g., degummed or substantially sericin free silk fibroin). In some embodiments, a silk material may be or comprise silk powder (e.g., comprising a plurality of silk particles).

In some embodiments, a silk fibroin material may be or comprise silk particles (e.g., microparticles or nanoparticles). As used herein, the term “particles” includes spheres, rods, shells, prisms, and related structures. While any application-appropriate particle size is contemplated as within the scope of the present disclosure, in some embodiments, a silk particle be have a diameter between 1 nm and 1,000 μm (e.g., between 1 nm and 1 μm, between 1 μm and 1,000 μm, etc). In some embodiments, a silk particle may have a diameter of greater than 1,000 μm.

Various methods of producing silk particles (e.g., nanoparticles and microparticles) are known in the art. For example, a milling machine (e.g., a Retsch planetary ball mill) can be used to produce silk powder. Generally, the ball mill consists of either two or four sample cups arranged around a central axis, which is geared such that each cup rotates both centrally and locally. Each ceramic cup is filled with small ceramic spheres. A range of sizes is available; balls with a diameter of 10 millimeters were/are used for the milling operations described in the present disclosure. As the cups spin, the spheres crush material in the cups to a small characteristic size. Both degummed and non-degummed silk can be converted from pulverized material to powder form in the ball mill.

In other embodiments, alternative powder formation techniques can be used (e.g., lyophilization or flash freezing and crushing). In other embodiments, alternative grates on the pulverizer, with larger holes, can be used. This can generate larger silk particle sizes.

In some embodiments, silk particles can be produced using a freeze-drying method as described in U.S. Provisional Application Ser. No. 61/719,146, filed Oct. 26, 2012, content of which is incorporated herein by reference in its entirety. Specifically, silk foam can be produced by freeze-drying a silk solution. The foam then can be reduced to particles. For example, a silk solution can be cooled to a temperature at which the liquid carrier transforms into a plurality of solid crystals or particles and removing at least some of the plurality of solid crystals or particles to leave a porous silk material (e.g., silk foam). After cooling, liquid carrier can be removed, at least partially, by sublimation, evaporation, and/or lyophilization. In some embodiments, the liquid carrier can be removed under reduced pressure. After formation, the silk fibroin foam can be subjected to grinding, cutting, crushing, or any combinations thereof to form silk particles. For example, the silk fibroin foam can be blended in a conventional blender or milled in a ball mill to form silk particles of desired size.

In some embodiments, the silk fibroin material comprising substantial amounts of amorphous structure is prepared from silk solution and is composed of nanostructures, an may be referred to as nano-sized silk powder (NSP) and be part of materials referred to amorphous silk nanomaterials (ASN). As used herein, these terms are equivalent and may be used interchangeably.

Without wishing to be held to a particular theory, in some embodiments, the present disclosure encompasses the recognition that the use of particular starting materials (e.g., silk fibroin material comprising substantial amounts of amorphous structure) allows for the production of previously unattainable compositions. In some embodiments, a silk material is not made from solubilized silk. In some embodiments, a silk material may be lyophilized.

### Silk Fibroin

According to various embodiments, any silk fibroin may be used in provided methods. In some embodiments, the silk fibroin is selected from the group consisting of spider silk (e.g., from Nephila ciavipes), silkworm silk (e.g., from Bombyx mori), and recombinant silks (e.g., produced/engineered from bacterial cells, yeast cells, mammalian cells, transgenic animals, and/or transgenic plants).In accordance with various embodiments, silk used in provided methods and compositions is degummed silk (i.e. silk fibroin with at least a portion of the native sericin removed). Degummed silk can be prepared by any conventional method known to one skilled in the art. For example, B. mori cocoons are boiled for a period of pre-determined time in an aqueous solution. Generally, longer degumming time generates lower molecular silk fibroin. In some embodiments, the silk cocoons are boiled for at least 60 minutes, at least 70 minutes, at least 80 minutes, at least 90 minutes, at least 100 minutes, at least 110 minutes, at least 120 minutes, or longer. Additionally or alternatively, in some embodiments, silk cocoons can be heated or boiled at an elevated temperature. For example, in some embodiments, silk cocoons can be heated or boiled at about 101.0° C., at about 101.5° C., at about 102.0° C., at about 102.5° C., at about 103.0° C., at about 103.5° C., at about 104.0° C., at about 104.5° C., at about 105.0° C., at about 105.5° C., at about 106.0° C., at about 106.5° C., at about 107.0° C., at about 107.5° C., at about 108.0° C., at about 108.5° C., at about 109.0° C., at about 109.5° C., at about 110.0° C., at about 110.5° C., at about 111.0° C., at about 111.5° C., at about 112.0° C., at about 112.5° C., at about 113.0° C., 113.5° C., at about 114.0° C., at about 114.5° C., at about 115.0° C., at about 115.5° C., at about 116.0° C., at about 116.5° C., at about 117.0° C., at about 117.5° C., at about 118.0° C., at about 118.5° C., at about 119.0° C., at about 119.5° C., at about 120.0° C., or higher. In some embodiments, such elevated temperature can be achieved by carrying out at least portion of the heating process (e.g., boiling process) under pressure. For example, suitable pressure under which silk fibroin fragments described herein can be produced are typically between about 10-40 psi, e.g., about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 31 psi, about 32 psi, about 33 psi, about 34 psi, about 35 psi, about 36 psi, about 37 psi, about 38 psi, about 39 psi, or about 40 psi.

In some embodiments, the aqueous solution used in the process of degumming silk cocoons comprises about 0.02M Na₂CO₃. The cocoons are rinsed, for example, with water to extract the sericin proteins. The degummed silk can be dried and used for preparing silk powder. Alternatively, the extracted silk can dissolved in an aqueous salt solution. Salts useful for this purpose include lithium bromide, lithium thiocyanate, calcium nitrate or other chemicals capable of solubilizing silk. In some embodiments, the extracted silk can be dissolved in about 8M-12 M LiBr solution. The salt is consequently removed using, for example, dialysis.

In some embodiments, the silk fibroin is substantially depleted of its native sericin content (e.g., 5% (w/w) or less residual sericin in the final extracted silk). In some embodiments, the silk fibroin is entirely free of its native sericin content. As used herein, the term “entirely free” (i.e. “consisting of” terminology) means that within the detection range of the instrument or process being used, the substance cannot be detected or its presence cannot be confirmed. In some embodiments, the silk fibroin is essentially free of its native sericin content. As used herein, the term “essentially free” (or “consisting essentially of”) means that only trace amounts of the substance can be detected, is present in an amount that is below detection, or is absent.

If necessary, a silk solution may be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of about 10% to about 50% (w/v). A slide-a-lyzer dialysis cassette (Pierce, MW CO 3500) can be used. However, any dialysis system can be used. The dialysis can be performed for a time period sufficient to result in a final concentration of aqueous silk solution between about 10% to about 30%. In most cases dialysis for 2-12 hours can be sufficient. See, for example, International Patent Application Publication No. WO 2005/012606, the content of which is incorporated herein by reference in its entirety. Another method to generate a concentrated silk solution comprises drying a dilute silk solution (e.g., through evaporation or lyophilization). The dilute solution can be dried partially to reduce the volume thereby increasing the silk concentration. The dilute solution can be dried completely and then dissolving the dried silk fibroin in a smaller volume of solvent compared to that of the dilute silk solution. In some embodiments, a silk fibroin solution can optionally, at a suitable point, be filtered and/or centrifuged. For example, in some embodiments, a silk fibroin solution can optionally be filtered and/or centrifuged following the heating or boiling step. In some embodiments, a silk fibroin solution can optionally be filtered and/or centrifuged following the dialysis step. In some embodiments, a silk fibroin solution can optionally be filtered and/or centrifuged following the step of adjusting concentrations. In some embodiments, a silk fibroin solution can optionally be filtered and/or centrifuged following the step of reconstitution. In any of such embodiments, the filtration and/or centrifugation step(s) can be carried out to remove insoluble materials. In any of such embodiments, the filtration and/or centrifugation step(s) can be carried out to selectively enrich silk fibroin fragments of certain molecular weight(s).

In some embodiments, silk fibroin and/or a silk fibroin article, may comprise a protein structure that substantially includes β-turn and/or β-strand regions. Without wishing to be bound by a theory, the silk β sheet content can impact gel function and in vivo longevity of the composition. It is to be understood that composition including non-β sheet content (e.g., e-gels) can also be utilized. In some embodiments, silk fibroin has a protein structure including, e.g., about 5% β-turn and β-strand regions, about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of these embodiments, silk fibroin has a protein structure including, e.g., at least 10% β-turn and β-strand regions, at least 20% β-turn and β-strand regions, at least 30% β-turn and β-strand regions, at least 40% β-turn and β-strand regions, at least 50% β-turn and β-strand regions, at least 60% β-turn and β-strand regions, at least 70% β-turn and β-strand regions, at least 80% β-turn and β-strand regions, at least 90% β-turn and β-strand regions, or at least 95% β-turn and β-strand regions. In yet other aspects of these embodiments, silk fibroin has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions. In some embodiments, silk β sheet content, from less than 10% to ˜ 55% can be used in the silk fibroin compositions disclosed herein.

In some embodiments, silk fibroin, or a silk fibroin article, has a protein structure that is substantially-free of α-helix and/or random coil regions. In aspects of these embodiments, the silk fibroin has a protein structure including, e.g., about 5% α-helix and/or random coil regions, about 10% α-helix and/or random coil regions, about 15% α-helix and/or random coil regions, about 20% α-helix and/or random coil regions, about 25% α-helix and/or random coil regions, about 30% α-helix and/or random coil regions, about 35% α-helix and/or random coil regions, about 40% α-helix and/or random coil regions, about 45% α-helix and/or random coil regions, or about 50% α-helix and/or random coil regions. In other aspects of these embodiments, the silk fibroin has a protein structure including, e.g., at most 5% α-helix and/or random coil regions, at most 10% α-helix and/or random coil regions, at most 15% α-helix and/or random coil regions, at most 20% α-helix and/or random coil regions, at most 25% α-helix and/or random coil regions, at most 30% α-helix and/or random coil regions, at most 35% α-helix and/or random coil regions, at most 40% α-helix and/or random coil regions, at most 45% α-helix and/or random coil regions, or at most 50% α-helix and/or random coil regions. In yet other aspects of these embodiments, the silk fibroin has a protein structure including, e.g., about 5% to about 10% α-helix and/or random coil regions, about 5% to about 15% α-helix and/or random coil regions, about 5% to about 20% α-helix and/or random coil regions, about 5% to about 25% α-helix and/or random coil regions, about 5% to about 30% α-helix and/or random coil regions, about 5% to about 40% α-helix and/or random coil regions, about 5% to about 50% α-helix and/or random coil regions, about 10% to about 20% α-helix and/or random coil regions, about 10% to about 30% α-helix and/or random coil regions, about 15% to about 25% α-helix and/or random coil regions, about 15% to about 30% α-helix and/or random coil regions, or about 15% to about 35% α-helix and/or random coil regions.

### Elevated Temperatures

As discussed herein, provided methods and compositions include the exposure to elevated temperature(s). As used herein, the term “elevated temperatures” refers to temperatures higher than standard room temperature (i.e., greater than 25° C.). In some embodiments, provided methods or compositions include exposure to a single elevated temperature. In some embodiments, provided methods or compositions include exposure to at least two elevated temperatures (e.g., 3, 4, 5, 6, 7, 8, 9, 10 or more). In some embodiments where a method of composition includes two or more elevated temperatures, at least two of those elevated temperatures are different from one another.

In some embodiments, an elevated temperature may be between 25° C. and 200° C. By way of specific exemplary ranges, in some embodiments, an elevated temperature may be between 25° C. and 150° C., between 25° C. and 100° C., between 25° C. and 95° C., between 25° C. and 50° C., between 50° C. and 200° C., between 50° C. and 150° C., between 50° C. and 100° C., between 25° C. and 100° C. . . . between 125° C. and 200° C., or any other range between 125° C. and 175° C.

In some embodiments, an elevated temperature may be at least 25° C. By way of additional example, in some embodiments, an elevated temperature may be at least 26° C., 27° C., 28° C., 29° C., 30° C., 35° C., 40° C., 45° C., 50° C., 55° C., 60° C., 65° C., 70° C., 75° C., 80° C., 85° C., 90° C., 95° C. or 100° C. In some embodiments, enhanced crystallization of silk fibroin material is observed at temperatures at or above 95° C.

In some embodiments, an elevated temperature may be at most 125° C. By way of additional example, in some embodiments, an elevated temperature may be at most 126° C., 127° C., 128° C., 129° C., 130° C., 135° C., 140° C., 145° C., 150° C., 155° C., 160° C., 165° C., 170° C., 175° C., 180° C., 185° C., 190° C., or 195° C.

Application of elevated temperature(s) to a provided composition or in a provided method may occur in any application-appropriate manner. By way of non-limiting example, in some embodiments, application of elevated temperature(s) may be via heat pressing, via a heating device such as an oven, heating stage, exposed flame or other mechanism.

Application of elevated temperature(s) may occur at or over any of a variety of time periods.

For example, in some embodiments, application of elevated temperature(s) occurs substantially instantly (e.g., by placement over a flame or in an oven). In some embodiments, application of elevated temperature(s) occurs over a period of seconds, minutes, or hours. In some embodiments, application of elevated temperature(s) occurs over a period of time between 1 second and 1 hour.

### Elevated Pressure

As discussed herein, provided methods and compositions include the exposure to elevated pressure(s). As used herein, the term “elevated pressures” refers to pressures higher than standard atmospheric pressure (i.e., 1.013 bar). In some embodiments, provided methods or compositions include exposure to a single elevated pressure. In some embodiments, provided methods or compositions include exposure to at least two elevated pressures (e.g., 3, 4, 5, 6, 7, 8, 9, 10 or more). In some embodiments where a method of composition includes two or more elevated pressures, at least two of those elevated pressures are different from one another.

Any application-appropriate method(s) may be used to cause elevated pressure as applied to provided compositions or in provided methods. By way of non-limiting example, in some embodiments, elevated pressure may include use of a vacuum, a press (e.g. heat press), and combinations thereof.

In some embodiments, application of elevated pressure may be or include uniaxial compression. In some embodiments, application of elevated pressure may be or include multi-axial compression (e.g., biaxial compression).

While any application-appropriate level of elevated pressure may be used, in some embodiments, an elevated pressure between 1MPa and 1GPa is used. By way of specific exemplary ranges, in some embodiments, an elevated pressure may be between 10MPa and 1GPa, between 50 MPa and 1GPa, between 100 MPa and 1GPa, between 200 MPa and 1GPa, between 300 MPa and 1GP, between 400 MPa and 1GPa or between 500 MPa and 1GPa. In some embodiments, an elevated pressure may be or comprise at least 1MPa (e.g., at least 2 MPa, 3 MPa, 4 MPa, 5 MPa, 6 MPa, 7 MPa, 8 MPa, 9 MPa, 10 MPa, 20 MPa, 30 MPa, 40 MPa, 50 MPa, 60 MPa, 70 MPa, 80 MPa, 90 MPa, 100 MPa 150 MPa, 200 MPa, 250 MPa, 300 MPa, 350 MPa, 400 MPa, 450 MPa, 500 MPa, 550 MPa, 600 MPa, 650 MPa, 700 MPa, or 750 MPa).

### Silk Articles

In some embodiments, provided silk articles exhibit a substantially homogenous structure. As used herein, “substantially homogenous structure” means that silk fibroin molecules are distributed and/or configured in a consistent way throughout substantially all of a portion of or the entirety of an article. Further, in some embodiments, silk articles may exhibit significant amounts of silk fibroin in a semi-crystalline structure. In some embodiments, production of a silk article according to provided methods includes a transition on the structure of silk fibroin from a substantially amorphous state to a semi-crystalline state, for example, as observed via X-ray diffraction.

In some embodiments, a silk article may exhibit significant amounts of B-sheet structure. For example, in some embodiments, a silk article may exhibit at least 10 wt % more (e.g., at least 20 wt %, 30 wt %, 40 wt %) B-sheet structures as compared to the starting silk fibroin material. In some embodiments, a silk article may exhibit at least 50 wt % more (e.g., at least 60 wt %, 70 wt %, 80 wt %, 90 wt %, 95 wt %) B-sheet structures as compared to the starting silk fibroin material.

In some embodiments, crystallinity of silk articles may be controlled by the application of temperature and pressure. For example, in some embodiments, when amorphous silk is processed at temperatures ranging from about 25° C.-125° C., the silk article may contain about 10-15% β-sheet structures. In some embodiments when amorphous silk is processed at temperatures ranging from about 125° C.-175° C., the silk article may contain for example, about 20-35% β-sheet structures or for example, over 40% β-sheet structures.

In some embodiments, provided methods and compositions allow for the production of silk articles which that are homogenous, where the silk amorphous powders are packed together via the bonding between neighboring raw silk powders, for example, at processing temperatures of about 25° C.-95° C. In some embodiments, provided methods and compositions allow for the production of silk articles that are homogenous, where the silk molecules of amorphous powders gain more mobility as they are heated above the glass transition temperature and self-assemble into interlocked nanoglobules, for example, at processing temperatures of about 125° C.-175° C.

In some embodiments, provided methods and compositions allow for the production of silk articles (e.g., thin films) that undergo thermal softening and are bendable and moldable into a desired shape. In some embodiments, provided methods and compositions allow for the production of silk articles that are machinable.

Provided methods and compositions allow for the production of complex silk articles in ways that were not achievable using previous methods (e.g., silk screws that can resist torsion forces relevant to in vivo use). By way of non-limiting example, in some embodiments provided methods and compositions may be used to produce silk articles such as films, fibers, meshes, needles, tubes, plates, screws, rods, and any combination thereof.

In some embodiments, a silk article may be amenable to one or more types of patterning. In some embodiments, patterning may be or comprise macropatterning. In some embodiments, patterning may be or comprise micropatterning (i.e., patterning with micro scale features). In some embodiments, patterning may be or comprise nanopatterning (i.e., patterning with nano scale features). In some embodiments, patterning may be or comprise: etching, lithography-based patterning, carving, cutting, and any combination thereof.

In some embodiments, a silk article may be subjected to one or more types of processing (e.g., 2, 3, 4, 5, 6, 7, 8, 9, 10 or more). While any application-appropriate form of processing is contemplated as within the scope of the present disclosure, in some embodiments, processing may be or comprise machining, rolling, drilling, milling, sanding, punching die cutting, extruding, chemical etching, coating, molding, turning, thread rolling, and any combination thereof.

### Exemplary Properties or Characteristics of Silk Articles

In some embodiments, provided compositions (e.g., silk articles) may be substantially transparent. In some embodiments, provided compositions (e.g., silk articles) may be semi-transparent. In some embodiments, provided compositions (e.g., silk articles) may be substantially non-transparent. As used herein, the term “transparent” refers to the propensity of an object to transmit light (with or without scattering of said light). In some embodiments, a composition/article is said to be substantially transparent if it transmits ≥80% of light it is exposed to in the visible range (400nm-800nm). In some embodiments, a composition/article is said to be semi-transparent if it transmits between 50%-80% of light it is exposed to in the visible range (400nm-800nm). In some embodiments, a composition/article is said to be substantially non-transparent if it transmits ≤50% of light it is exposed to in the visible range (400nm-800nm).

In some embodiments, provided compositions may be biocompatible and/or biodegradable. In some embodiments, provided compositions may exhibit particular degradation profile(s). By way of specific example, in some embodiments, a provided composition may degrade at least 50 wt % after about 96 hours of exposure to an aqueous environment at 37° C. In some embodiments, a provided composition may not degrade more than 10% after months of exposure to an in vivo environment or condition.

In some embodiments, provided compositions may exhibit one or more desirable properties including, but not limited to: electrical conductivity, enhanced machinability, and/or enhanced thermoformability.

### Additives

In some embodiments, provided methods and compositions include one or more additives (e.g., 2, 3, 4, 5, 6, 7, 8, 9, 10 or more). In some embodiments, at least one additive may be mixed with or otherwise associated with a silk fibroin material prior to an applying step (e.g. exposure to one or more of elevated temperature and elevated pressure). In some embodiments, at least one additive may be mixed with or otherwise associated with a silk fibroin material substantially at the same time as an applying step). In some embodiments, at least one additive may be mixed with or otherwise associated with a silk fibroin material subsequently to an applying step.

Provided methods and compositions are amenable to the addition of any of a variety of additives. By way of non-limiting example, in some embodiments an additive may be or comprise a small molecule, an organic macromolecule, an inorganic macromolecule, an electrically conductive material, an inorganic material, a hydrophobic material, a hydrophilic material, a nanomaterial, and any combination thereof.

The processing of the silk-based materials, including pure silk materials and silk-based composite materials, can be modified with addition of one or more additives. In some embodiments, a function of an additive may be to tune the processing conditions and the properties of the products. In some embodiments, additives may be selected from water; glycerol; saccharides; biological macromolecules, e.g. peptide, proteins; antibodies and antigen binding fragments; nucleic acids; immunogens; antigens; enzyme; synthetic polymers, e.g. poly(ethylene) glycol, poly-lactic acid, poly(lactic-co-glycolic acid) to name but a few specific examples, though any application-appropriate additive is specifically contemplated as within the scope of the present disclosure.

In some embodiments, for example some embodiments contemplated for in vivo use, provided compositions may comprise one or more proteases. In some embodiments, an organic macromolecule is or comprises at least one protease. In some embodiments, a protease is or comprises one or more of Proteinase XIV, Proteinase K, a-chymotrypsin, collagenase, matrix metalloproteinase-1 (MMP-1), and MMP-2. In some embodiments, a protease may be useful in tailoring the degradation profile of a particular provided composition (e.g., in an in vivo environment).

In some embodiments, an electrically conductive material may be or comprise an organic conductive material and/or an inorganic conductive material (e.g., a metal). In some embodiments, an electrically conductive material may be or comprise at least one of a conductive polymer, graphene, silver, gold, aluminum, copper, platinum, steel, brass, bronze, and iron oxide.

Any application-appropriate amount of one or more additives may be useful according to various embodiments. By way of non-limiting example, in some embodiments, an additive may be present in a provided composition in an amount between 0.001 wt % and 95 wt %. In some embodiments, one or more additives may be mixed with a silk fibroin material in an amount ranging between 0.001 wt % and 95 wt % of the silk fibroin material.

Disclosed herein is a new method to plasticize silk materials. In some embodiments, the method comprises two steps: 1) lyophilized silk powders (LSPs) are first exposed to an aqueous mist environment to introduce single or multiple plasticizers to the system homogeneously; 2) mist-treated LSPs are subject to compression molding to generate plasticized silk materials.

### EXAMPLES

Example 1: Preparation of regenerated silk powder—Bombyx mori cocoons were cut into small pieces and boiled in an aqueous 0.02 M Na2CO3 (Sigma-Aldrich) solution for 30 min, followed by rinsing in distilled water to remove the Na2CO3 and sericin. The degummed silk was then dried overnight and dissolved in 9.3 M LiBr at 60° C. for 4 hours, yielding a 20% (w/v) solution. The solution was subsequently dialyzed against distilled water for 2 days using dialysis tubing with a 3.5 kDa cutoff molecular weight. After dialysis, the solution was centrifuged for 20 min at 9,780 g twice to remove insoluble impurities. The silk concentration was determined by evaporating water from a solution of known weight and weighing the remaining solid using an analytical balance (˜6w/w %). The silk solution was diluted and frozen. For the glycerol plasticizer system, 10-30 wt % of glycerol was incorporated into the diluted silk solution, followed by frozen. The frozen silk solution or silk/glycerol solutions were then lyophilized at −80° C. and 0.006 bar until complete sublimation. The lyophilized silk or silk/glycerol was milled into fine powders using a high-speed analytical mill (20,000 rpm., 2 min, Col-Parmer), giving the product referred to as lyophilized silk powders or silk/glycerol powder. The powders were stored in ambient dry conditions to prevent any rehydration until used.

### Example 2: Plasticizing silk materials via mist treatment and compression molding

The silk powder or silk/glycerol powder was plasticized at 4° C. with the water mist treatment by a humidifier for a certain time to obtain the desired water contents before the further processing steps below. Plasticized silk materials were packed into the predesigned molds, followed by thermal pressing at 632 MPa with variable temperatures for 15-30 min.

### Example 3: Characterization

¹³C Solid-state Nuclear Magnetic Resonance (SS-NMR), X-ray diffraction (XRD), scanning electron microscopy (SEM), Thermal gravimetric analysis (TGA), differential scanning calorimetry (DSC) and mechanical testing were used to characterize the silk powder or silk/glycerol powder, plasticized silk powder and plasticizer-assisted thermal molding silk products. Details of some of the above methods are provided below.

Solid-state NMR experiments were performed on a Bruker AVANCE III HD 600 MHz spectrometer (Bruker BioSpin GmBH, Germany), with 13C and 1H resonance frequencies of 150.90 and 600.13 MHz, respectively. All experiments were conducted in a 4 mm CP/MAS broadband probe. A pulse sequence with a ramped (100-50) 1H-13C cross-polarization (CP) period followed by 1H-decoupled 13C detection was used. The contact time and 1H decoupling field strength were 1 ms and 69 kHz, respectively. The recycle delay was 3-4 s. All the experiments were conducted at ambient temperature. The magic angle spinning speed was 8 kHz for all samples. The chemical shift was calibrated using 1,4-di-tert-butylbenzene by setting the unprotonated carbon signal to 148.8 ppm. Deconvolution of 13C SSNMR spectra was performed using OriginLab software. Gaussian function was selected to fit the region of 13C SSNMR spectra to each peak assigned to Ala, Gly, and Ser residues over the region (12-70, and 165-180 ppm, respectively). Then, the fitted peak centers, peak area, and full width at half-maximum of each peak were obtained for further structure content determination.

The morphologies of the materials were characterized by Field-emission scanning electron microscopy (FE-SEM, Gemini 560, ZEISS). The SEM images were collected with a voltage of 5 kV, and the samples were prepared by sputtering a 20 nm layer of Pt/Pd.

Differential Scanning calorimetry (DSC) and Thermogravimetric Analysis (TGA) of materials were measured under a nitrogen atmosphere by using DSC Q20 and TGA Q50 (TA Instruments, US) systems. The material (3-5 mg) was weighed in an aluminum pan and equilibrated at the desired relative humidity.

The thermal degradation of the materials was characterized by TGA heated at 10° C./min from ambient temperature to 500° C. in triplicate. DSC measurements of the materials were carried out at the scan rate of 10° C./min from −50 to 200° C. under a dry nitrogen gas flow of 50 ml min⁻¹.

X-ray diffraction (XRD) structures of the samples were investigated by using a Rigaku SmartLab system (Rigaku Corporation, Japan) with CuKα radiation (λ=1.5418 Å) at room temperature. The diffractometer is equipped with a two-dimensional SC-70 detector, which was operated in 1D mode. The XRD measurements were performed in a parallel beam setting, in the 2θ range 5-50° with a step size of 0.01° and speed of 2°/minute. The voltage and current settings were 40 kV and 44 mA, respectively.

The tensile tests were measured on an Instron 5565 machine (Instron, USA) in tensile test mode at 25° C. and 45% relative humidity with a loading rate of 1 mm min⁻¹. The tested bulk silk bioplastics were in plate format with a length of 10 mm and a width of 5 mm. Multiple samples (>=3) were tested for each condition.

Example 4: Silk screw fabrication: Silk bone screws were machined from silk bars using a CNC lathe (Trak TRL 1440 EX, Southwestern Industries). A custom single point external cutter (Vargud) was used on the CNC lathe to cut screw threads by matching the turning speed with the horizontal speed of the cutter to cut a desired pitch length (outer diameter ˜1.8 mm, pitch 600 μm). The screw heads were machined to have a cylindrical head, and a slot was generated.

### Example 5: Molecular Dynamics Simulation

The initial model structure of silk was constructed based on the theoretically predicted structure deposited in the Protein Data Bank (PDB ID: 2SLK). The model was first subjected to the 200 ns conventional molecular dynamics (MD) simulation under the Generalized Born implicit solvent model. In the simulation, the Born radius was set to 12 Å, and the ion concentration was 0.05 M. The topology and force field parameters of the protein were obtained from the CHARMM36m force field parameter set. The simulation temperature was set to 25° C. by a Langevin thermostat. All bond lengths involving hydrogen atoms were constrained by the SHAKE algorithm. The integration step is 2 fs. In this stage, the representative protein structure of the equilibrated MD trajectory was used for the following MD calculations.

To correspond to our experimental conditions, two types of simulation structures were remodeled. One type was silk protein, while the other was the protein structure with 452 TIP3P water molecules (a water mass ratio of 20%). Before MD simulations, 50,000 steps of conjugation gradient minimization were performed to release some high-energy contact in the protein structures. After that, the equilibrium MD simulations of 200 ns were carried out to obtain stable structures under the isothermal-isobaric ensemble. The simulation temperature in the stage was kept at 25° C. by a Langevin thermostat, and the pressure of the systems was adjusted to 1 atm by the modified Nosé-Hoover Langevin piston method. Next, the simulation conditions in the production phase were set to the following circumstances. Under anhydrous conditions, the temperature values were set to 25° C. As water containing silk model, the temperatures were set to 4° C. and 60° C. with a high pressure of 625 MPa, respectively. For each simulation condition, a 200 ns MD simulation was performed to explore the conformational changes of the silk protein. All the MD simulations were implemented by the NAMD 2.14 package. In the simulations, periodic boundary condition were used. The short-range nonbonded interactions were turned off in the distance range from 10 to 12 Å. The pair list distance was set to 14 Å. The long-range electrostatic interactions were calculated by the PME method. VMD and PyMOL were used for the visualization of the simulation trajectories. The protein conformation was analyzed by the STRIDE method.

### Example 6: Cell Culture

C2C12 mouse skeletal myoblast cells (ATCC, USA) were cultured in Dulbecco's Eagle Medium (ThermoFisher Scientific, USA) supplemented with 10% fetal bovine serum (ThermoFisher Scientific, USA) and 1% antibiotic-antimycotic (ThermoFisher Scientific, USA) in standard culture conditions, 37° C. and 5% CO2 humidified atmosphere. Five million C2C12s at passage 4-8 were deposited onto each silk micropillar for cell seeding experiments. The silk micropillars with cells were incubated at 37° C. and 5% CO2 humidified atmosphere for 1-3 h to allow cell adhesion, followed by transfer to 24-well plates for further cell culture.

The activity of C2C12s on the silk micropillars was investigated by AlamarBlue (ThermoFisher Scientific, USA). The silk micropillars with cells were incubated in 10% AlamarBlue reagent at 37° C. and 5% CO2 humidified atmosphere for 2 h. The fluorescence of the incubated AlamarBlue solutions was recorded with a microplate reader (Varioskan™ LUX multimode, ThermoFisher Scientific, USA) with excitation/emission at 560/590 nm. Silk micropillars with cells were fixed in 4% paraformaldehyde (ThermoFisher Scientific, USA) for 30 min at room temperature and permeabilized with 0.1% Triton X-100 (ThermoFisher Scientific, USA) in PBS for 40 min, followed by blocking in 3% bovine serum albumin (BSA, Millipore Sigma, USA) in PBS for 30 min. The samples were incubated with myosin heavy chain antibody with 2.5 μg/mL (ThermoFisher Scientific, USA) in blocking solution at 4° C. overnight and were stained by secondary antibodies (1:400 Goat anti-Mouse Alexa Fluor™ 594 and 1:400 Alexa Fluor™ 488 Phalloidin, ThermoFisher Scientific, USA) and 1:500 DAPI (ThermoFisher Scientific, USA) in 0.3% BSA blocking solution at room temperature for 1 h. The samples were then washed three times in PBS for imaging. A confocal laser scanning microscope (SP 8, Leica, Germany) was used for image acquisition.

### Results

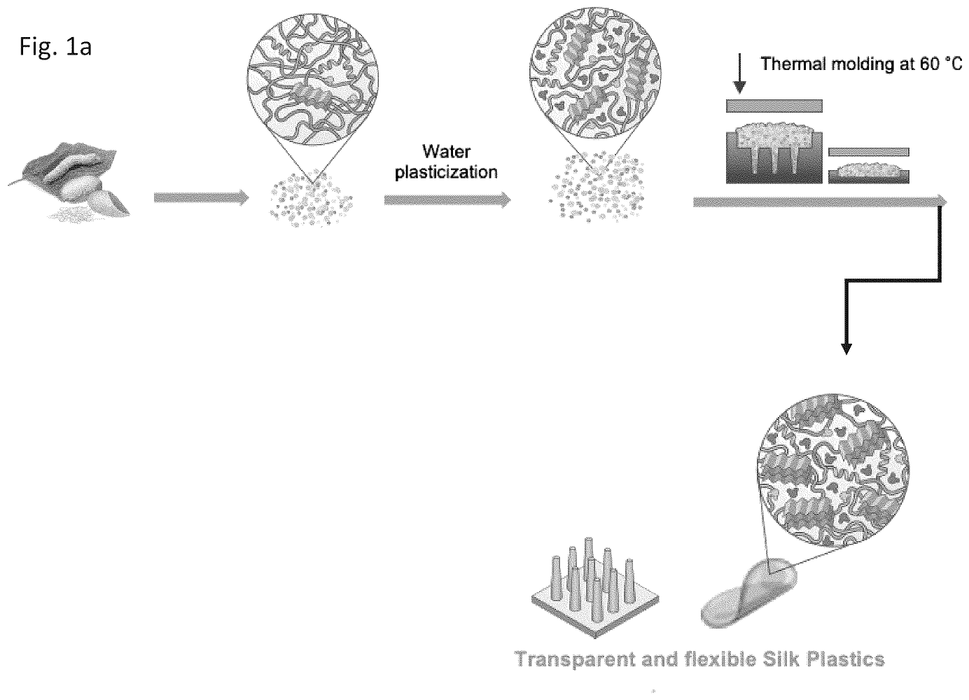

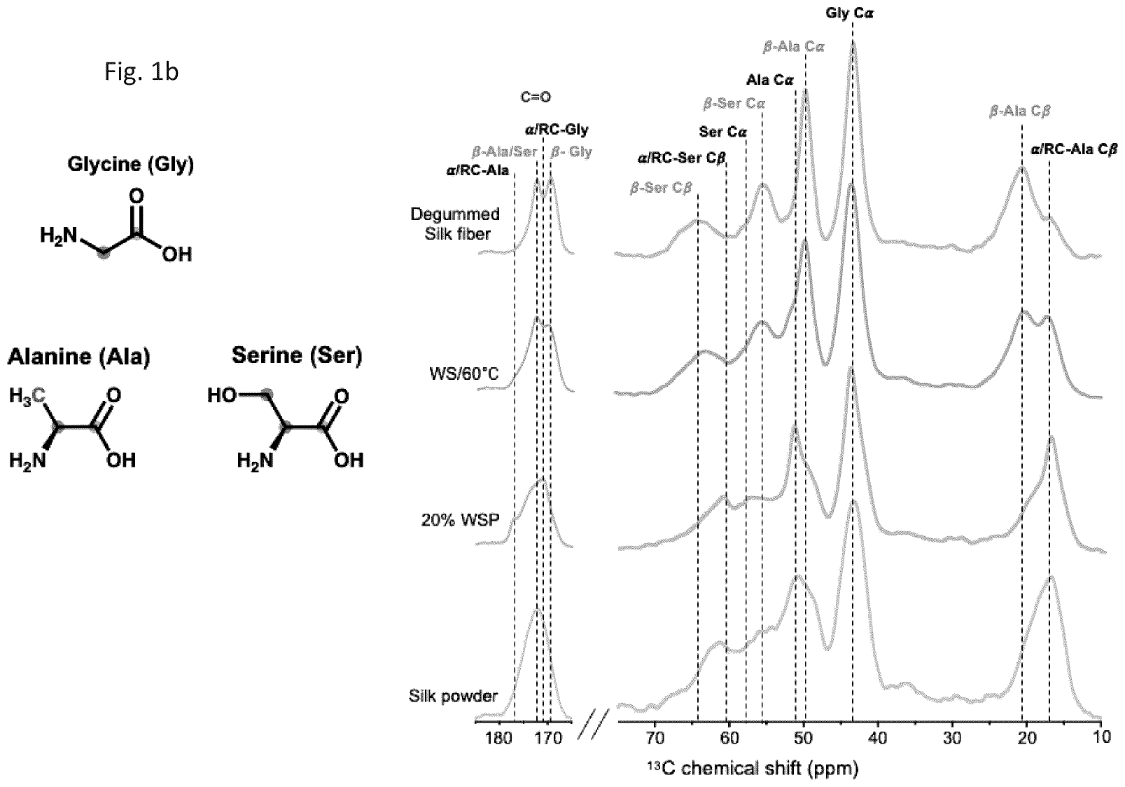

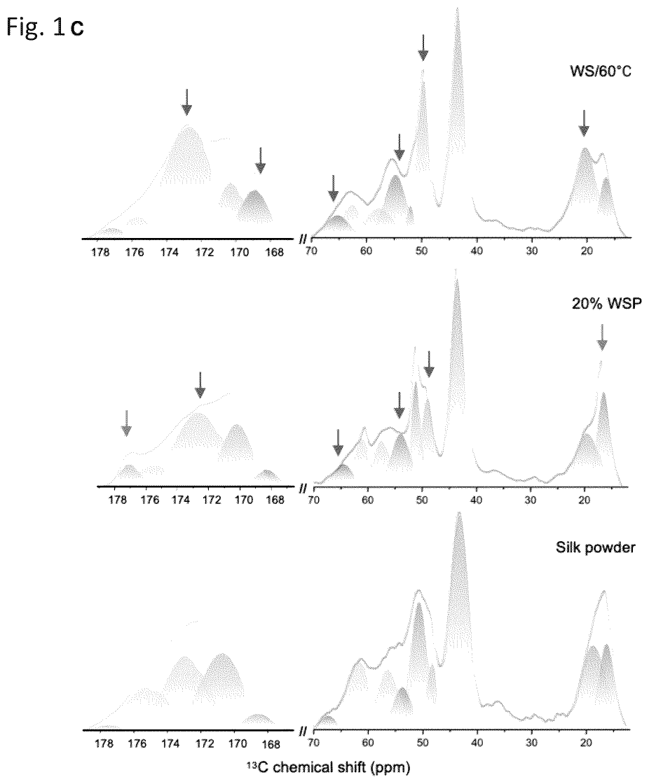

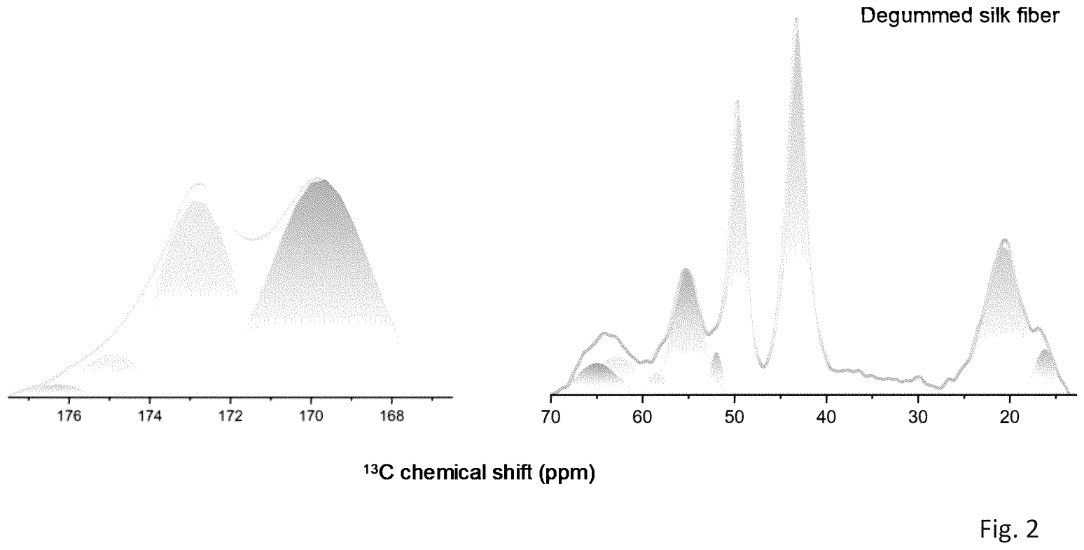

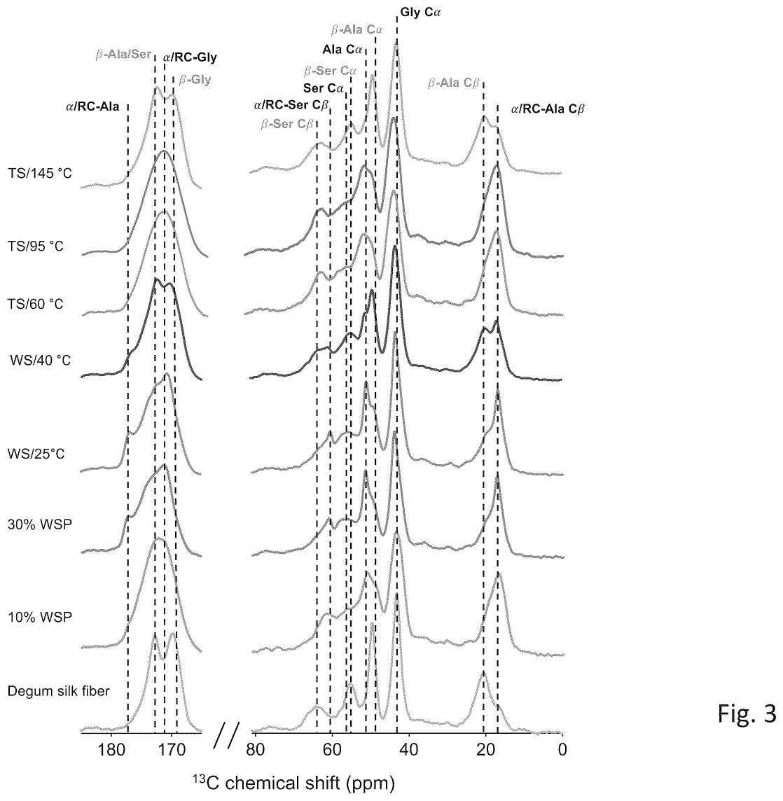

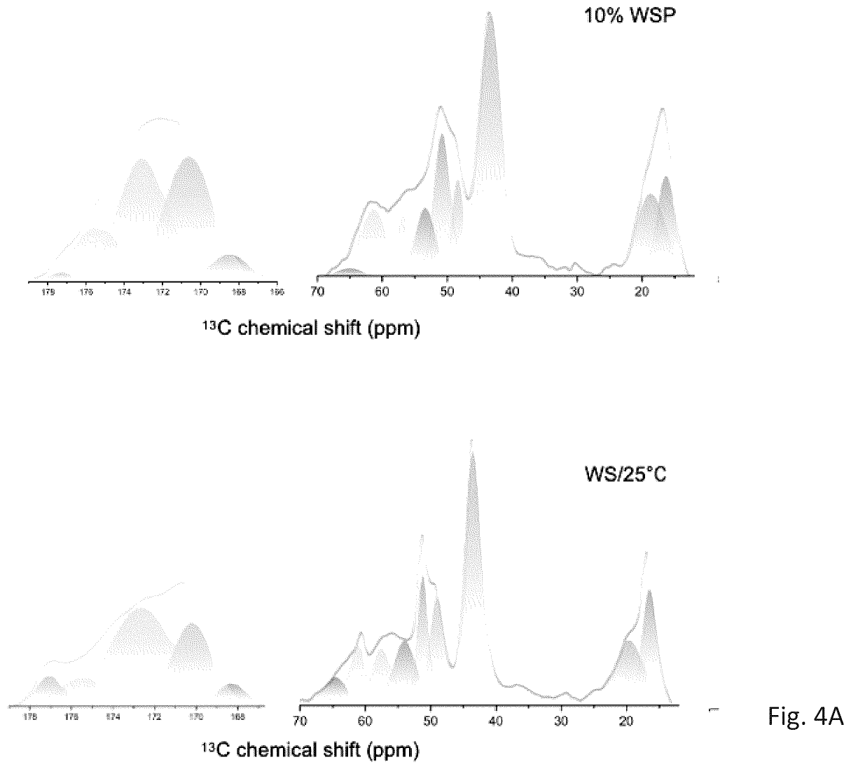

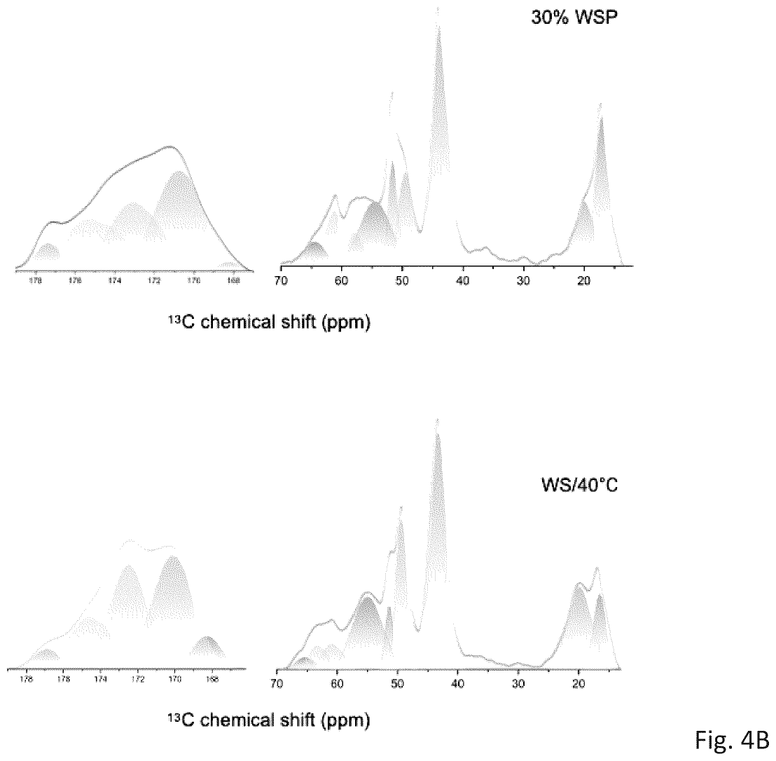

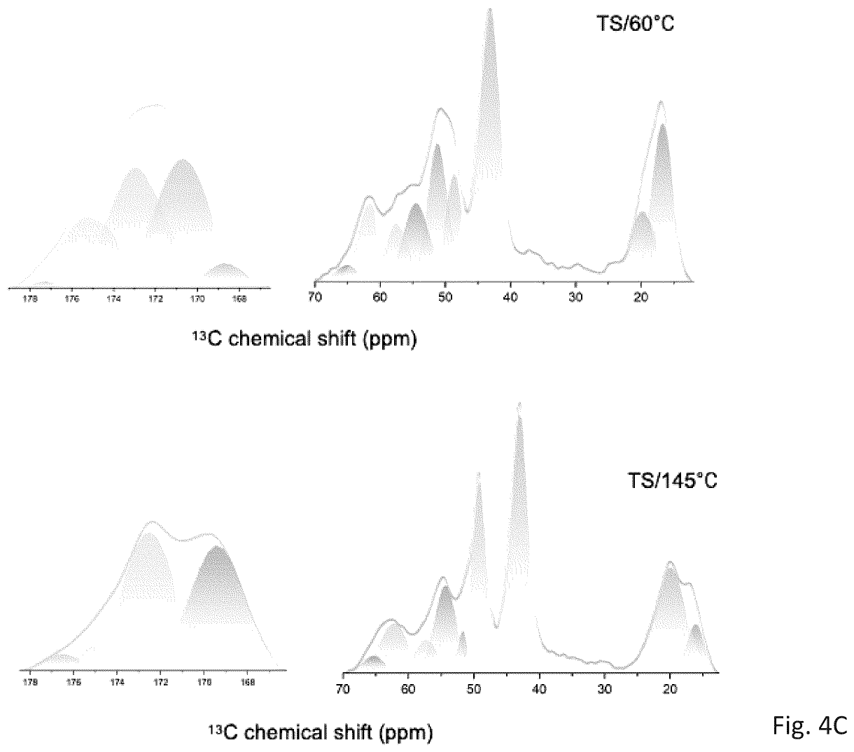

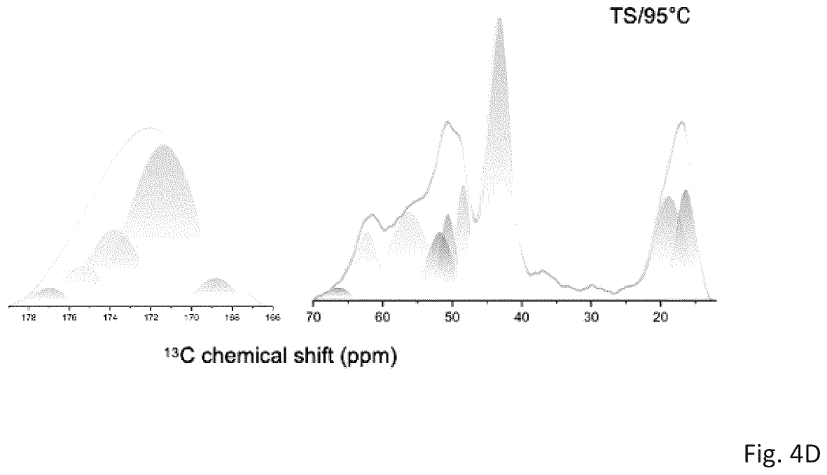

Transparent and flexible silk bioplastics with variable shapes can be molded via a plasticizer-assisted thermal molding process. As shown in FIG. 1a, the lyophilized silk powder as the raw materials was loaded into predesigned molds after water mist plasticization treatment, followed by compressing the powder at 60° C. with a pressure of 632 MPa; this method of using free water molecules as the plasticizer enhanced mobility of the amorphous silk protein chains allowing the silk to transform and mold from amorphous to crystalline-dominated structures with a higher β-sheet content due to the process, and providing flexibility to the final silk bioplastics. ¹³C Solid-state nuclear magnetic resonance (SSNMR) was utilized to identify and quantify the structural forms and proportions of the silk powder, water-plasticized silk powder with 20% of water content (20% WPS), 20% WPS thermal-molded at 60° C. (WS/60° C.), and degummed native silk fibers, respectively, with the degummed native silk fibers serving as reference or control materials. The SSNMR spectra of the samples are presented in FIG. 1b, with the main signals as the Cα and Cβ carbons of Ala and Ser, Gly Cα, and C═O peaks, respectively, and the deconvolution of all the peaks is shown in FIG. 1c and FIG. 2. Based on previous studies, the peaks at 20.3, 49.6, 55, 64.5,169, and 172.7 ppm were assigned to β-sheet conformations of silk and the structures dominated by β-sheet are attributed to the silk II form, while the other signals were characterized as random coils, the silk I form. Other intermediates were categorized as silk I-like forms for simplicity. The water plasticization of the silk powder resulted in a narrowing of the peak at 16.6 ppm and a pronounced shoulder of the carbonyl peak at 177.1 ppm, indicating a decrease in silk I-like forms and peak shifts to fields assigned to β-sheet conformations at 49.6, 55, 64.5, and 172.7 ppm. Significant structural transitions to secondary silk II occurred with the thermal molding of water-plasticized silk powders at 60° C., evidenced by an increased area fraction of peaks at 20.3, 49.6, 55, 64.5, 169, and 172.7 ppm. Furthermore, the effects on the conformational transitions throughout the plasticizer-assisted thermal molding process were quantitatively evaluated by deconvolution of the SSNMR spectra of silk powder with different water contents and molded at various temperatures (FIGS. 3 and 4A-D). The dry-state silk powder was fused and densified with silk II structure formation by direct thermal molding without plasticization, as the bound water enhanced the mobility of the amorphous regions of the silk. However, under these conditions the increments of β-sheet content were limited to the dry powder molded at 60 and 95° C. (TS/60° C. and TS/95° C.), and the β-sheet content increased when the molding temperature was raised to 145° C. (TS/145° C.).

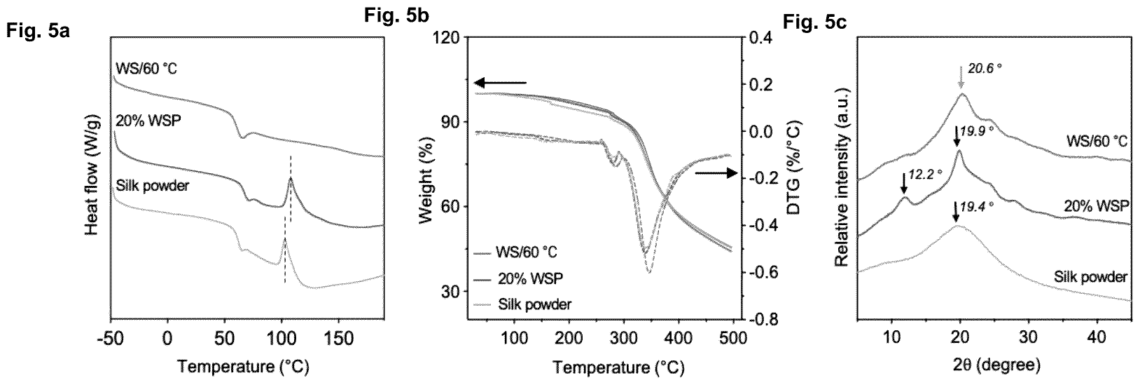

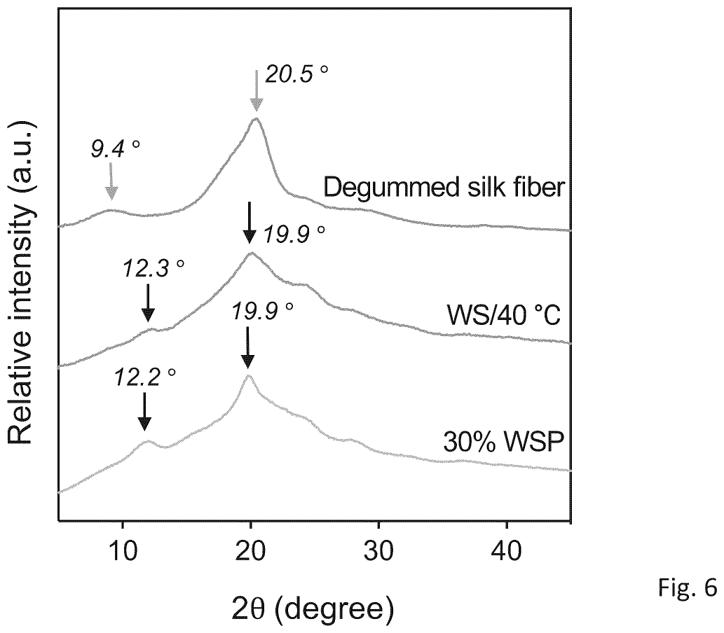

Thermal analysis and X-ray diffraction analysis (XRD) were performed to reveal correlations within the thermal stability and crystalline structure transitions of the silk bioplastics during plasticization and thermal molding. As shown in FIG. 5a, all endothermic peaks below 100° C. were detected in the differential scanning calorimetric curves (DSC) and were derived from the loss of water, while the exothermic peaks were attributed to β-sheet crystallization of the silk powder. The lyophilized 20% WPS were detected between 100° C. and 110° C., demonstrating that the position of the crystallization peak shifted to higher temperatures after the plasticization with water. In addition, no exothermic peak for crystallization was detected for WS/60° C., suggesting that plasticization and thermal molding induced β-sheet crystallization, and thermal stability was enhanced with this increased crystallinity. Thermogravimetric (TGA) curves in FIG. 5b reflect the same trend as found with the DSC results, indicating that WS/60° C. with high β-sheet crystallization exhibited improved thermal stability and the lowest weight loss among the samples. XRD results also provided insight into the dynamic conformational transitions of the silk bioplastics. With the broad peak at 19.4° for the amorphous silk powder and peaks at 9.4° and 20.5° identified for typical silk II appearing in the degummed silk fibers as reference, the 20% WPS curve with the silk I associated peak at 12.2° confirmed that the plasticization facilitated an amorphous-silk I transition. The following molding step promoted silk II crystallinity increases as the temperature reached 60° C., as evidenced by the peak at 20.5° of the WS/60° C. (FIG. 5c and FIG. 6).

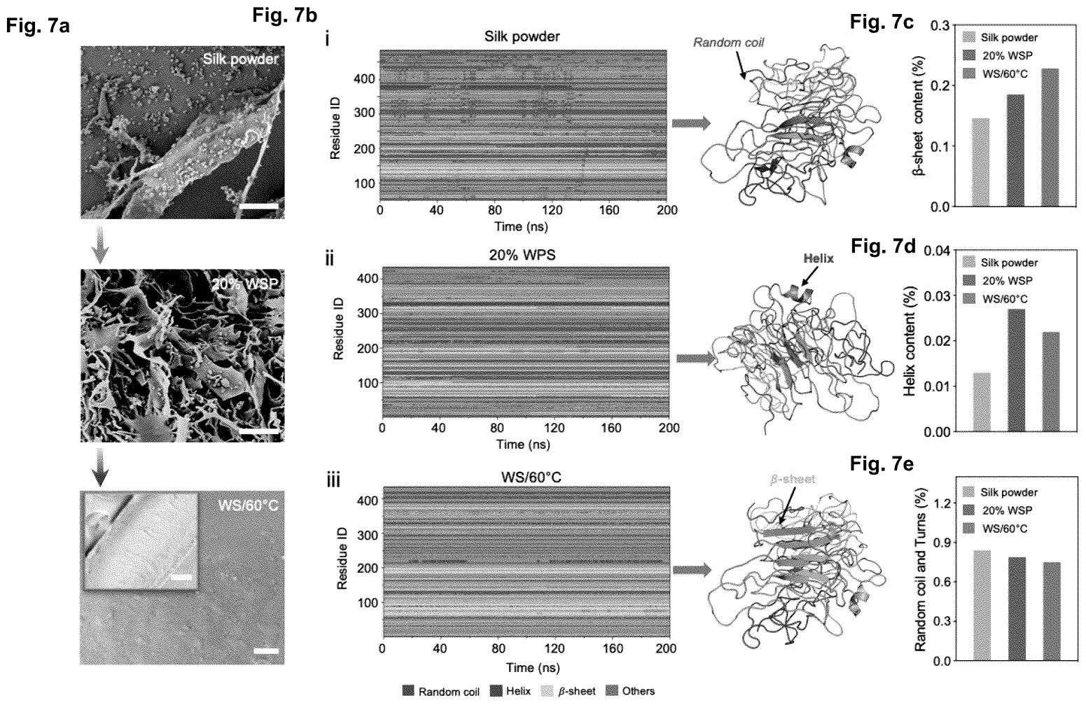

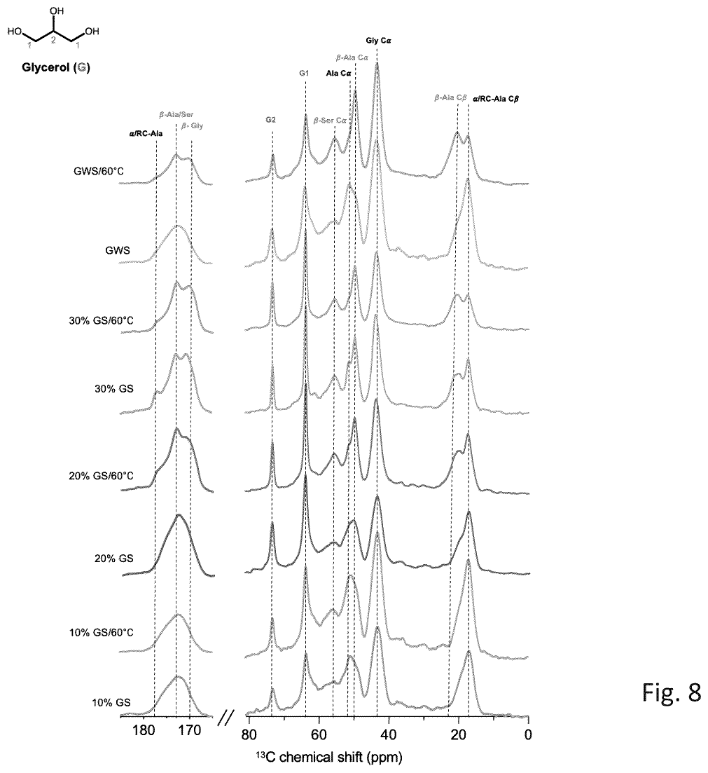

Cross-sectional scanning electron microscopy (SEM) images display the information on the microstructural transformation of these silk bioplastics (FIG. 7a). The loose silk powder particles were partially fused during the plasticization process, forming sheet-like and fibrous structures of 20% WPS. Subsequently, smooth and flat surfaces appeared in the low magnification SEM images of WS/60° C., indicating that further compaction and fusion from thermal molding led to cohesive and homogeneous structural transitions and through the assembly of nanoscale silk particles. To better understand crystalline transitions during plasticization and thermal molding, amino acid residues 152-586 in the silk protein sequence (UniProt ID: P05790) were selected for the construction of the silk powder model, and 20% water was added to the silk powder model as 20% WPS. The molecular dynamics simulations were investigated for the silk powder, 20% WPS and 20% WPS at 60° C. (WS/60° C.) models, respectively, and the dynamic secondary structural changes attributed to each amino acid residue were analyzed over a 200 ns simulation process (FIG. 7b). The structural changes of each single residue gradually stabilized for each model, and the trajectory patterns of these three models indicated that WPS 20% and WS/60° C. exhibited more stable and increased secondary structure distribution compared to the silk powder. Further, the β-sheet fraction of WS/60° C. increased, as shown in FIG. 7b (i-iii). Additionally, the corresponding representative snapshots of the silk powder, 20% WPS, and WS/60° C. presented in FIG. 7b represent a similar profile with their residue trajectory patterns. Moreover, the average content of β-sheet, helix, random coil, and other intermediate in the three models were quantified over the entire 200 ns simulation trajectory (FIG. 7c-e). Compared with the silk powder, the proportion of random coil and other intermediate structures in 20% WPS and WS/60° C. decreased, while WPS 20% and WS/60° C. exhibited the highest content of helix and β-sheet structure, respectively. Therefore, the simulation results revealed that the structural transitions of these three models was from metastable random coil structures to stable and ordered secondary structures dominated by helices and β-sheets during plasticization and thermal molding, which supports the experimental results above. To further exploit the plasticization effects for the silk thermal molding system, glycerol and a combination of water and glycerol were incorporated as the extended plasticizers for the silk structural transformation studies. The SSNMR spectra in FIG. 8 with the main signals corresponding to the carbons peaks of Ala, Ser, Gly, C═O, and glycerol, respectively, indicating that glycerol as a plasticizer shows the consistent effect of water on the structural transformation of silk proteins, which further proves the applicability of the plasticizer-assisted thermal molding method.

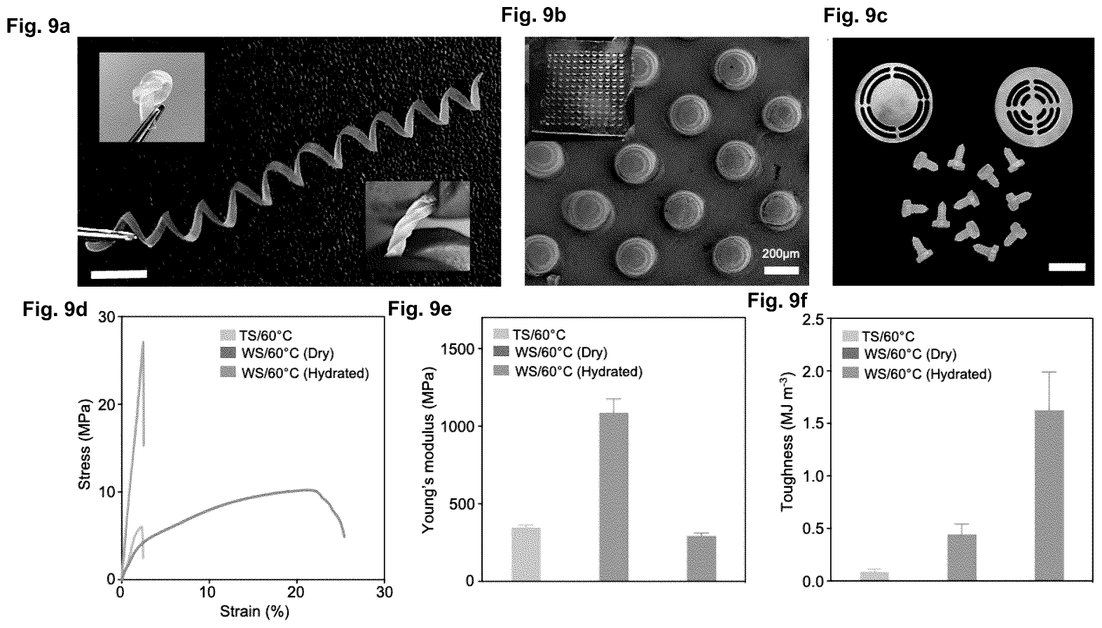

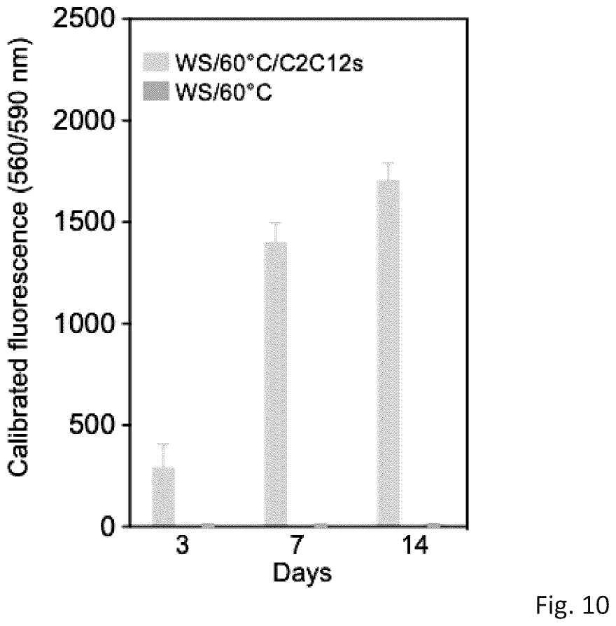

The water molecules in the amorphous regions improved protein chain mobility, providing flexibility to the materials, which allowed for the WS/60° C. to be very pliable (FIG. 9a), and also capable of fabrication of micropillars at the micron scale (FIG. 9b). The stiffness of the dehydrated silk samples was increased due to the high crystallinity, enabling machining into bone screws and lenses (FIG. 9c). Tensile tests were conducted on the WS/60° C. in both dry and hydrated states for mechanical characterization. As shown in FIG. 9d-f, the β-sheet dominated structures demonstrates that the dry WS/60° C. exhibited improved stiffness with tensile modulus increased from 0.35 to 1.08 GPa when compared to the TS/60° C. In contrast, the hydrated WS/60° C. was more extensible with the highest tensile strain to 20% and average tensile toughness of 1.62 MPa m⁻³, resulting from the enhanced mobility of the amorphous protein chains. Thus, stiffness and ductility were realized with the silk bioplastics simultaneously, which provides options for mechanical characteristics tailored with plasticizer-assisted thermal molding. Silk micropillars with a geometry of 200 μm height, 200 μm radius, and 250 μm adjacent distance were used as substrates for the culture of C2C12s for two weeks. Cell activity and immunofluorescence images acquired by confocal laser scanning microscopy (CLSM) indicated that the attachment and continuous proliferation of the C2C12 cells on the silk micropillar substrates during the two weeks of culture, comparing to the silk micropillars without cells seeding as the control (Applicant can provide color figures illustrating this effect to a patent examiner, upon request; see also FIG. 10 depicting fluorescence density data). In addition, myotubes labeled by MF 20 were observed in the CLSM images, suggesting cell differentiation induced by the silk micropillar pattern (Applicant can provide color figures illustrating this effect to a patent examiner, upon request).

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

1. A method of making a silk fibroin article, the method comprising:
a) mist-plasticizing a lyophilized silk fibroin powder, thereby producing a modified powder, wherein the mist-plasticizing comprises exposing the lyophilized silk fibroin powder to a mist of an aqueous plasticizer composition; and
b) thermally compressing the modified powder into a solid form, thereby forming the silk fibroin article.

2. The method of claim 1, the method further comprising lyophilizing a silk fibroin solution to form the lyophilized silk fibroin powder.

3. The method of claim 1, wherein the mist-plasticizing of step a) is performed at a temperature of between 0° C. and 25° C.

4. The method of claim 1, wherein the aqueous plasticizer composition is a plasticizer solution including plasticizer in an amount by weight of between 0.1% and 50%.

5. The method of claim 1, wherein the thermally compressing of step b) is performed at a temperature of between 1° C. and 165° C., between 1° C. and 95° C., between 1° C. and 65° C., between 1° C. and 50° C., or between 1° C. and 30° C.

6. The method of claim 1, wherein the thermally compressing of step b) is performed at a pressure of between 100 MPa and 1000 MPa.

7. The method of claim 1, wherein the thermally compressing of step b) is applied for a length of time of between 1 second and 10 minutes.

8. The method of claim 1, the method further comprising reducing the size of the silk fibroin article using a manual or automated tool.

9. The method of claim Error! Reference source not found., [symbol] wherein the manual or automated tool is a lathe, a saw, a drill, a file, sandpaper, or a combination thereof.

10. The method of claim 1, wherein the mist density of the mist-plasticizing of step a) is selected for a desired material property in the silk fibroin article.

11. A silk fibroin article made by the method of claim 1.

12. A silk fibroin material having a solid-state NMR ¹³C spectrum having a C═O-associated signal with at least some peak splitting and an alanine β-carbon-associated signal with at least some peak splitting, wherein an alpha/RC portion of the alanine β-carbon-associated signal associated with alpha-helix and random coil structures has a peak intensity that is higher than a beta portion of the alanine β-carbon-associated signal associated with beta sheet structures.

13. The silk fibroin material of claim Error! Reference source not found., [symbol] wherein a beta portion of the C═O-associated signal associated with beta sheet structures has a peak intensity that is higher than an alpha/RC portion of the C═O associated signal associated with alpha-helix and random coil structures.

14. A thermally compressed silk fibroin article formed from a mist-plasticized lyophilized silk fibroin powder.
